(19) United States
(12) Patent Application Publication
(10) Pub. No.: US 2006/0062833 A1
(43) Pub. Date: Mar. 23, 2006

(54) Wound dressing

(75) Inventor: Derek Moe, Maple Grove, MN (US)

Correspondence Address: CIMA;LERNER, DAVID ET AL, 600 SOUTH AVENUE WEST, WESTFIELD, NJ, 07090, US

(73) Assignee: CIMA LABS INC., Eden Prairie, MN (US)

(21) Appl. No.: 11/227,630

(22) Filed: Sep. 15, 2005

Related U.S. Application Data
(60) Provisional application No. 60/611,984, filed on Sep. 22, 2004.

(51) Int. Cl. A61L 15/00

(52) U.S. Cl. 424/445

(57) ABSTRACT

The present invention relates to a wound dressing that can be applied as a solid to a wound area and can assist in stopping blood flow and promoting clot formation.

### CROSS-REFERENCE TO RELATED APPLICATIONS

This application claims the benefit of the filing date of U.S. Provisional Patent Application No. 60/611,984 filed Sep. 22, 2004, the disclosure of which is hereby incorporated herein by reference.

### BACKGROUND OF THE INVENTION

Medafor, Inc., having an office at 2700 Freeway Boulevard, Suite 800, Minneapolis, Minn. 55430 sells a number of products based on their MPH™ technology. “MPH” stands for microporous polysaccharide hemospheres. One of their products, sold under the trade name TraumaDEX, is a powdered topical wound dressing for control of severely bleeding wounds from traumatic injuries including cuts, lacerations and puncture wounds. According to the company's website, MPH™ affects near instantaneous hemostasis at wound sites, even in the presence of profuse bleeding. The technology consists of engineered biopolymer microporous particles with a controlled pore size, which are designed to act as a sieve to dehydrate the blood and thus serve to accelerate the natural clotting process. Using this technology, clotting has been demonstrated to initiate within as little as 30 seconds to one minute, compared to as much as 30 minutes required using traditional hemostats. Applied topically, TraumaDEX powder gels rapidly with no tissue irritation, creating a protective environment for hemostasis and healing. The particles or beads are derived from plant-based biomaterials (believed to be starches) that have an extensive history of use in humans. MPH™ materials are bioinert, contain no human or animal proteins, are stable and require no mixing prior to application.

However, the TraumaDEX material is a powder and can be difficult to use in a number of environments. Trying to apply a powder outdoors or in a windswept environment can be quite difficult. This is particularly true when wound dressing is occurring during a time of stress such as immediately after an accident or during a battle. A patient must keep sufficiently still to allow the powder to gel and begin clotting in the wound area. The patient, or someone assisting the patient, must be steady in their application of the powder to ensure that enough material actually is applied to the wound as necessary. Moreover, an arm or a toe for example is rounded and it may be difficult to place sufficient powder in contact with the afflicted area.

If this type of wound dressing material could be formulated in a solid, non-powdered form having a defined shape and size, the ease of application and therefore the usefulness of this type of wound dressing material would be greatly enhanced.

### SUMMARY OF THE INVENTION

The invention includes a solid wound dressing having a defined shape and hardness comprising: an anhydrous mixture of a clot forming agent, a binder and a compression lubricant.

In another embodiment, the present invention provides a solid wound dressing comprising: a compressed anhydrous mixture of a clot forming agent, a binder, a water soluble filler and a compression lubricant having a defined size and shape. The solid wound dressing preferably has a thickness of about 1 inch or less and a dimension of about 3 inches or less.

In another embodiment, the present invention provides a wound dressing kit comprising: a solid wound dressing having a defined size and shape comprising a compressed anhydrous mixture of a clot forming agent, a binder, a water soluble filler, and a compression lubricant. The wound dressing preferably has a thickness of about 0.5 inches or less and a dimension of about 3 inches or less and is packaged in a blister package.

In a particularly preferred embodiment, the blister package is a non-push through type that includes a manually delaminable backing layer which is peeled away to release the solid wound dressing from the package. In another preferred embodiment, the blister is a bubble such as that disclosed in U.S. Pat. No. 6,155,423.

A method of treating a wound is also part of the invention and comprises the steps of: removing a solid wound dressing having a defined size and shape comprised of an anhydrous mixture of a clot forming agent, a binder and a compression lubricant from a delaminable blister package by peeling back a backing layer thereof; and applying the solid wound dressing to a wound to thereby initiate clotting and scab formation in the wound.

### BRIEF DESCRIPTION OF THE FIGURES

FIG. 1 is a prospective view of a solid wound dressing in accordance with one aspect of the present invention.

FIG. 2 is a prospective view of a solid wound dressing in accordance with another aspect of the present invention.

FIG. 3 is a prospective view of a solid wound dressing in accordance with one aspect of the present invention.

FIG. 4 is a sectional view of a blister package and solid wound dressing in accordance with the invention.

### DETAILED DESCRIPTION

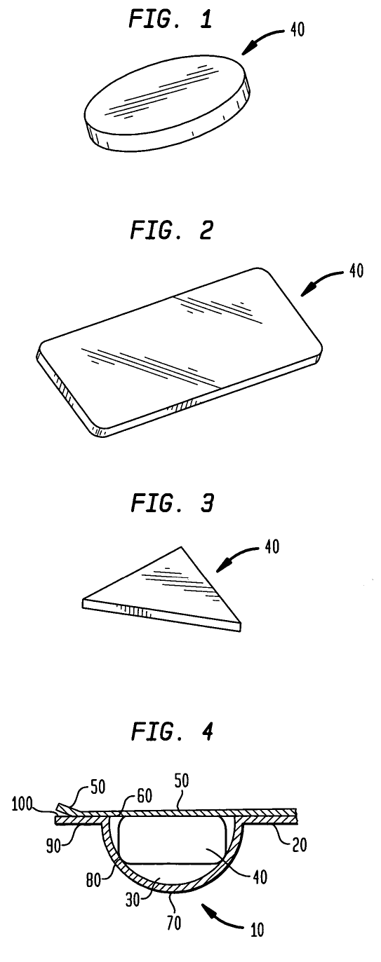

The word “solid” as used in connection with a solid wound dressing in accordance with the present invention does not encompass powders or particulate per se. Although such materials are solid (as opposed to, for example, liquid or gas), and are used to make the wound dressings of the invention, they are not shaped three-dimensional articles as the term solid encompasses in accordance with the invention. The solid in accordance with the present invention should be understood to encompass shaped three-dimensional articles. “Defined shape” in accordance with the present invention means that the solid wound dressing has a particular desired three-dimensional shape. This can be a cylindrical wafer having a specified radius and thickness, as shown in FIG. 1, can be in the form of a generally square or oblong bar or ingot as shown in FIG. 2 or any polygon, such as a triangle, as shown in FIG. 3. The shape may be very important to applying the solid wound dressing of the present invention to particular areas of the body. However, as long as the shape is predefined having a certain thickness and, depending upon its shape, a radius, length and wide, etc., it qualifies as a defined shape.

“Anhydrous” in accordance with the present invention means that the resulting solid wound dressing, when packaged, should have a residual moisture content of less than about 5%, more preferably less than about 2%.

A “clot forming agent” in accordance with the present invention can be any material capable of being formed into a solid wound dressing and which, when applied to a wound, can act as a molecular sieve and absorb certain fluids in the blood. Preferably, proteins and cellular constituents can adhere to its surface thus forming a compacted mass which accelerates the normal clotting cascade. Preferred clot forming agents include those sold by Medafor, Inc. under the trade name TraumaDEX™.

Other clot forming agents may include starch, such as corn starch, modified starches including, without limitation, starch graft copolymers, carboxymethyl starch and the like, cross-linked PVP, croscarmellose salt, silicates such as calcium silicate, cross-linked HPC, microcrystalline cellulose and the like. Without wishing to be bound by any particular theory of operation, it is believed that these materials absorb at least the blood plasma within a few moments, helping to stop the flow of blood, desiccating the area and promoting the formation of clots. Certain formulations in accordance with the present invention may in addition form a gel or other more fluid or more malleable substance, which can adapt to the size and contour of the wound and help as a bandage or plug. In some instances, at least a portion of these materials may actually form a portion of the clot and assist in protecting the wound and in healing. Many materials that are compressible may be used in a system described herein and can operate in either of the manners described herein are contemplated.

A “binder” in accordance with the present invention can be any binder which is nontoxic and which is capable of providing adhesion to the solid wound dressing of the present invention. Particularly preferred binders are binders known in the pharmaceutical industry used typically in the formation of tablets. These include, for example, microcrystalline cellulose and starch. A particularly preferred form of microcrystalline cellulose useful in accordance with the present invention is a low moisture content material such as that sold under the trademark AVICEL PH 113.

A “compression lubricant” in accordance with the present invention may not be necessary if the solid wound dressing of the present invention is not formed by a compression-like technique. For example, such a lubricant may not be necessary if the materials in question are mixed with a liquid solvent or liquid binder into a mold and allowed to harden. However, where compression will be used to manufacture the solid wound dressing, such as using a tablet press or like technique, or compression molding, it is preferred that some form of compression lubricant be applied. Compression lubricants can be applied externally to the mold or die used to form the solid wound dressing. However, this can be cumbersome in high speed compression techniques. Far more useful, therefore, is the mixture of a compression lubricant into the wound dressing formulation prior to compression. Any lubricant which can assist in the release of the solid wound dressing from the mold, die or punch following a compression step may be used. A preferred group of such compression lubricants are tableting lubricants or ejection lubricants known in the pharmaceutical industry. These include, for example, stearic acid, calcium stearate, talc and magnesium stearate.

The amount of clot forming agent in accordance with the present invention will depend upon a number of factors including the size and type of the wound contemplated, the extent of bleeding expected and, in particular, the other materials with which it will be mixed. Generally, as a minimum, sufficient clotting forming agent must be provided to allow for effective clot formation. The upper limit of the amount of clot forming agent is far less critical. However, the upper limit will generally be the maximum amount that the remaining portion of the formulation can carry effectively. By this it is understood that many traditional tableting excipients are known to be able to carry a certain load, i.e., they can carry a certain amount of drug and still form a cohesive tablet. The carrying capacity of a system will depend upon its components and their relative proportions. Thus the upper limits of the amount of clot forming agent in accordance with the present invention is the carrying capacity of the remaining materials.

Generally, however, the amount of clot forming material will range from between about 156 to about 75% by weight, more preferably between about 20% to about 60% by weight. Binders are typically used in an amount of between about 5% and about 30% by weight, more preferably between about 10% and about 20% by weight. Lubricants can be used in an amount of as little as 0.5% up to about 5% by weight. However, more preferably, the amount of lubricant will range from between about 1% and about 5% by weight.

It is also possible and often desirable in accordance with the present invention to use a filler. “Filler” in accordance with the present invention will be water soluble and preferably rapidly water soluble. In addition, the filler must be pharmaceutically acceptable. Fillers useful in accordance with the present invention include those typically used as fillers and diluents in the production of tablets and include sugars and sugar alcohols. Preferred fillers include mannitol, lactose, dextrose, sucrose, glucose, galactose, maltitol, maltodextrin, fructose, sorbitol and xylitol.

It is not generally important that these materials have a sweet taste. Although, as the solid wound dressing of the present invention may be applied to the inner surfaces of, for example, the mouth to assist in clot formation, it may be desirable that they be pleasant tasting. Sugars and sugar alcohols with a relatively high degree of sweetness or with, for example, a negative heat of solution, can provide additional organoleptic benefits. The amount of filler used can vary widely. It can range from between about 0% to about 70% and more preferably between about 5% to about 60%.

Other traditional excipients used in the pharmaceutical industry in the production of tablets may also be used. These can include colorings, flavorings, preservatives, glidants and disintegrants. These will be used in traditional amounts used in the industry and generally the total of these ingredients should not exceed approximately 20% by weight of the solid dosage form.

In a preferred embodiment, the solid wound dressings of the present invention can be produced by the methods typically used for forming tablets in the pharmaceutical industry. These include the use of generally high speed tablet presses. Materials can be blended, with or without such pretreating steps as granulation, and the materials fed into the hopper of a high speed multitablet press. These materials can then be compressed into a cohesive mass of defined dimensions. In cross-section, they may be round, oval shaped, or have the shape of any polygon. Indeed, they may form irregular shapes as well.

Generally the thickness of the solid wound dressing in accordance with the present invention will be less than one inch. More preferably it will be half an inch or less. It is also preferred that the solid wound dressings of the present invention have at least one dimension that is about three inches or less. By this it is understood that if a circular cross-section is used as shown in FIG. 1, the diameter of the circle will be three inches or less. Where an oblong shape is used, as in FIG. 2, one of the length or width will be three inches or less. If triangular as in FIG. 3, a line drawn from any angle to an opposite side, preferably such that the line is perpendicular to that side, will be three inches. It is of course possible, however, to form solid wound dressings in accordance with the present invention in any dimension desired.

It is also possible to manufacture solid wound dressings in accordance with the present invention by manually pouring a mixture of the materials in question into a mold and compressing them. Alternatively, a solvent, preferably a nonaqueous solvent, can be added such that a solution, slurry or dispersion can be formed and the resulting material poured into a mold of suitable size. Then the solvent can be evaporated. Additional binding materials may be useful in this sort of process.

It is important the wound dressings in accordance with the present invention rapidly mix with blood in the wound and begin to dissolve so as to form a gel or other structure so as to fill the wound and begin the clotting process. For that reason, it is important that the filler used be rapidly soluble such that it does not interfere with the process. Similarly, it is important that the amount of the remaining materials be controlled so that they do not unduly interfere with the speed at which the clotting process can take place. For the same reason, it is desirable that the solid wound dressings of the present invention be relatively soft and friable. This assists in promoting the rapid absorption of mixture and the transformation into a clotting mass.

Hardness in accordance with the present invention generally can range from about 10 up to about 100 Newtons. However, preferably, the hardness will range somewhere between about 10 and about 50 Newtons. Similarly, the wound dressings in accordance with the present invention can have any friability. Thus, they can have a friability of less than 2% where desirable. However, in a preferred embodiment, the friability is greater than 2%, when measured by standard USP techniques published in the U.S. Pharmacopoeia as of the filing date of this document.

It is also important that the wound dressing of the present invention be anhydrous prior to use. This can be accomplished by packaging in any number of formats. However, a particularly preferred format is a blister package. Blister packages are easy to use and lightweight, convenient and can provide some protection from breakage during storage and handling. While any blister package can be used, a particularly preferred blister package is that disclosed in U.S. Pat. No. 6,155,423, to Katzner et al., assigned on its face to CIMA LABS INC. of Eden Prairie, Minn., the text of which in its entirety including its explanation of how such blister packages are made, is hereby incorporated by reference.

A particularly preferred packaged solid wound dressing in accordance with one aspect of the present invention comprises a blister package 10 formed by a blister sheet 20 defining one or more recesses 30 in which wound dressings 40 are disposed and a sheet of lidding material 50 overlying the recesses 30 to cover the wound dressings 40 therein. Each recess 30 has an open top 60, a closed bottom 70 remote from the top, and walls 80 extending between the open top 60 and the bottom 70. The wound dressing 40 disposed in each recess 30 engages the walls 80 of each recess 30 so that the walls 80 hold the wound dressing 40 away from the bottom 70 of the recess 30 and adjacent the lidding material 50. This aspect protects the wound dressing 40 from damage by preventing shifting of the wound dressing 40 during transport. An empty space between each wound dressing 40 and the bottom 70 of the recess 30 in which the wound dressing 40 is disposed cushions the wound dressing 40 from impact when the package 10 is dropped. The sheet of lidding material 50 is peelably attached to the blister sheet 20 so that a user of the package 10 may peel back the lidding material 50 to gain access to the wound dressings 40. Note that in this embodiment, wound dressing 40 has completely rounded edges.

The blister sheet 20 of the packaged wound dressing 40 may define a flange 90 surrounding the open top 60 of each recess 30 and a generally planar top surface 100 facing in an upward direction. Where a flange is provided, the lidding material sheet 50 is peelably attached to the flange 90 of the blister sheet 20 so that the lidding material sheet 50 overlies the wound dressings 40 in each recess 30.

The packaged wound dressing may be comprised of a blister sheet having a plurality of recesses containing wound dressings arranged, for example, in rows and columns. In this example, each flange associated with each recess is substantially coplanar with and connected to adjacent flanges and the sheet of lidding material covers the plurality of flanges. Thus, the blister package in one embodiment includes a plurality of unit packages, each unit package incorporating one recess, a portion of the lidding sheet overlying that recess, and the flange associated with that recess. A set of tear lines is included between the flanges of adjacent unit packages so that a user of the package may tear along the tear lines to separate a unit package.

The recesses of the package and the wound dressings disposed in the recesses may have essentially any shape. For example, the wound dressing may be disk-shaped, oblong, square-shaped, triangular, octagonal and the like. Shapes for recesses include, without limitation, circular, oblong or polygonal to match the shape of the wound dressing.

Furthermore, the walls and bottom of the recesses may define a shape in the form of a surface of revolution, about a vertical axis normal to the flange surrounding each of the recesses. For example, the recesses may have a curved, cup-like bubble shape as shown in FIG. 4. Where the solid wound dressings are disc-shaped, they may each have an edge which contacts the walls of the recess in which each solid wound dressing is disposed. The edge and walls define an annular region of contact coaxial with the vertical axis of the recess. The edge of such a disc-shaped wound dressing may comprise a bevel which contacts the walls of the recess. The annular region of contact prevents shifting of the wound dressing within the blister and the damage to the wound dressing associated with such shifting.

To further ensure that fragile wound dressings are not damaged upon opening of the package, the packaged wound dressings may further comprise indicia on the blister package directing the user not to push the bottom of the recesses to eject a wound dressing from the package. Because some of the prior art packages are of the push-through type, users of packages may attempt to push a frangible wound dressing through the lidding material sheet by collapsing the blister sheet recess in which the wound dressing is disposed. Indicia provided on the package provides additional protection of the wound dressing against damage. The indicia may be visible from a downwardly-facing bottom surface of the blister sheet and may be printed on the bottom surface of the blister sheet.

A blister package in accordance with preferred aspects of the invention includes a unitary blister sheet defining a plurality of unit package regions. Each package region of the blister sheet has a recess, in which a solid wound dressing may be disposed, with an open top and a flange surrounding the recess. A unitary sheet of lidding material is peelably sealed to the flanges of the package regions for covering dosage forms which may be disposed in the recesses. Thus, the blister package includes, in this embodiment, a plurality of unit packages, each unit package incorporating one unit package region of the blister sheet and the portion of the lidding sheet which overlies that unit package region. The sheet of lidding material has lines of weakness between adjacent unit package regions so that each unit package is separable from the blister package. The lines of weakness have perforations and spaces between perforations. The lines of weakness cross each other to define intersections at corners of the unit packages. The lines of weakness intersect at the spaces, as opposed to the perforations, of the lines of weakness. These spaces form a dimple at the intersections when the package is torn along the tear lines to separate the unit packages.

Unsealed areas aligned with the intersections of the lines of weakness may be provided at a corner of each unit package. The unsealed areas provide a portion of lidding material on the corner of a separated package unit which can be grabbed by a user. The user may then peel back the lidding on the unit package to obtain access to a dosage form which may be disposed in the recess of the unit package. The blister sheet may be recessed below the flanges of the unit package in the corner unsealed areas to provide a separation of the blister sheet and lidding material for easier opening by the user. Prior to obtaining access to the unsealed area, the user must separate the lid and blister at the dimple at the intersection between the unit packages. This dimple hides the unsealed area from a child who has torn the package along the perforations.

Any type of blister package backing may be used. These include so-called push-through backings where, by application of pressure to the solid wound dressing, through the blister portion of the package, the wound dressing can force the rupture of and be pushed through the backing layer. However, because of the generally soft and friable nature of wound dressings in accordance with some preferred embodiments of the present invention, a non-push through backing is preferred. These backings are generally delaminable when a tab, generally a portion of the backing which has not been glued or otherwise adhered to another layer, at least in a local area, is grasped and peeled away to reveal the solid wound dressing within the well or lumen of the blister. To use the solid wound dressings of the present invention one grasps the blister package in question, lifts the tab on the backing material to cause delamination, revealing the wound dressing, removes the wound dressing from the package and applies it to the area in question, generally holding it in place for a time sufficient to allow bleeding to begin to stop and clotting to begin. The wound dressing may also include an adhesive layer which allows it to adhere directly to the wound area which can be coated on one or more of its sides. Any adhesive coating used in the formation of, for example, transdermal patches, may be used. Alternatively, a suitable bandage, pressure bandage or wrapping can be wrapped over same to hold it in place. The wound dressing, and/or any suitable bandage, for example, including same may be sterilized by known methods or may be non-sterile.

### EXAMPLE 1

Weigh out all items shown in the table below except for magnesium stearate and pass through 20 mesh screen. Add screened materials to V-Blender and mix for 30 minutes. Weigh out magnesium stearate and pass through a 20 mesh screen. Add the screened magnesium stearate to the mixed materials from step 2. Blend the combined materials together for 5 minutes in the V-Blender.

Tablet size=⅝″; Tablet weight (mg)=700 mg; tablet to a target hardness of (N)=30 N.

| | % w/w per | | g/130 g |
|---|---|---|---|
| Material | tablet | mg/Tablet | Batch |
| TraumaDex | 28.60 | 200.20 | 37.2 |
| Mannitol 60 | 27.45 | 192.15 | 35.7 |
| Mannitol EZ | 27.45 | 192.15 | 35.7 |
| Avicel PH113 | 15.00 | 105.00 | 19.5 |
| Magnesium Stearate | 1.50 | 10.50 | 2.0 |
| TOTAL: | 100.00 | 700.00 | 130.0 |

A Globe Pharma Minipress was used to compress tablets made as described above, with different compression forces used for examples one and two, while maintaining the weight the same.

### EXAMPLE 2

Weigh out all items listed in the table of Example 1 except for magnesium stearate and pass through 20 mesh screen. Add screened materials to V-Blender and mix for 30 minutes. Weigh out magnesium stearate and pass through a 20 mesh screen. Add the screened magnesium stearate to the mixed materials from step 2. Blend the combined materials together for 5 minutes in the V-Blender. Tablet to hardness of 20 N using procedure set forth in Example 1.

### EXAMPLE 3

Weigh out all items shown in the table below except for magnesium stearate and pass through 20 mesh screen. Add screened materials to V-Blender and mix for 30 minutes. Weigh out magnesium stearate and pass through a 20 mesh screen. Add the screened magnesium stearate to the mixed materials from step 2. Blend the combined materials together for 5 minutes in the V-Blender.

Tablet size=⅝″; Tablet weight (mg)=700 mg; Tablet to a target hardness of (N)=30 N.

| | % w/w per | | g/130 g |
|---|---|---|---|
| Material | tablet | mg/Tablet | Batch |
| Crospovidone | 38.50 | 269.50 | 115.5 |
| Mannitol 60 | 25.00 | 175.00 | 75.0 |
| Mannitol EZ | 25.00 | 175.00 | 75.0 |
| Avicel PH113 | 10.00 | 70.00 | 30.0 |
| Magnesium Stearate | 1.50 | 10.50 | 4.5 |
| TOTAL: | 100.00 | 700.00 | 300.0 |

An SMI Piccola tablet press was used to compress tablets made as described above, run at 35 rpm, with a small amount of pre-compression, in addition to the main compression.

Testing of the tablets of examples 1, 2 and 3 can be made by visual observation, which will show that in a dish with 2 mL of water, the tablet rapidly absorbs the water, leaving both a substantially dry dish and an intact tablet that has expanded in size.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A solid wound dressing having a defined shape and hardness comprising an anhydrous mixture of a clot forming agent, a binder and a compression lubricant.

2. The solid wound dressing of claim 1 wherein said clot forming agent is a microporous polysaccharide bead.

3. The solid wound dressing of claim 1 wherein said hardness is at least about 10 Newtons.

4. The solid wound dressing of claim 3 wherein said hardness ranges from between about 10 Newtons to about 50 Newtons.

5. The solid wound dressing of claim 1 having a friability of at least about 2% when measured by USP.

6. The solid wound dressing of claim 1 wherein said clot forming agent is present in an amount which ranges from about 15% to about 85% by weight of said solid wound dressing.

7. The solid wound dressing of claim 6 wherein said clot forming agent is present in an amount which ranges from about 20% to about 60% by weight of said solid wound dressing.

8. The solid wound dressing of claim 6 wherein said binder is present in an amount which ranges from about 5% to about 30% by weight of said solid wound dressing.

9. The solid wound dressing of claim 6 wherein said compression lubricant is present in an amount which ranges from about 0.50%. to about 5% by weight of said solid wound dressing.

10. A solid wound dressing having a defined size and shape comprising: a compressed anhydrous mixture of a clot forming agent, a binder, a water soluble filler and a compression lubricant, said solid wound dressing having a thickness of about 0.50 inches or less and a dimension of about 3 inches or less.

11. The solid wound dressing of claim 10 wherein said water soluble filler is a sugar or sugar alcohol, present in an amount which ranges from about 5% to about 60% by weight of said solid wound dressing.

12. The solid wound dressing of claim 11 wherein said sugar or sugar alcohol is selected from mannitol, lactose, dextrose, sucrose, glucose, galactose, maltitol, maltodextrin, fructose, sorbitol or xylitol.

13. A wound dressing kit comprising a solid wound dressing having a defined size and shape comprising a compressed anhydrous mixture of a clot forming agent, a binder, optionally a water soluble filler, and a compression lubricant and having a thickness of about 0.50 inches or less and a dimension of about 3 inches or less packaged in a blister package.

14. The kit of claim 13 wherein said blister package contains a non-push through backing layer.

15. The kit of claim 13 wherein said blister package includes a manually delaminable backing layer which can be peeled away to release said dose and wound dressing from said package.

16. The kit of claims 14 or 15 wherein said water soluble filler is a sugar or sugar alcohol present in an amount ranging from about 0% to about 70% by weight of said solid wound dressing.

17. The kit of claim 16 wherein said wound dressing has a hardness of at least about 10 Newtons or a friability of at least about 2% when measured by USP.

18. The kit of claim 17 wherein said clot forming agent is present in an amount which ranges from about 15% to about 85%, and said binder is present in an amount which ranges from about 5% to about 30% by weight of said solid wound dressing.

19. A method of treating a wound comprising the steps of: removing a solid wound dressing having a defined size and shape comprised of an anhydrous mixture of a clot forming agent, a binder and a compression lubricant from a delaminable blister package by peeling back a backing layer thereof; and applying said solid wound dressing to a wound to thereby initiate clotting and scab formation in said wound.
